(19) United States
(12) Patent Application Publication
(10) Pub. No.: US 2022/0392646 A1
(43) Pub. Date: Dec. 8, 2022

(54) METHOD AND SYSTEM FOR RECOMMENDATION OF DISEASE-RELATED RESOURCES

(71) Applicant: KONINKLIJKE PHILIPS N.V., EINDHOVEN (NL)

(72) Inventors: Mengzhu XU, SHANGHAI (CN); Junzeng FU, SHANGHAI (CN); Mengzhe TAO, SHANGHAI (CN); Yi ZHOU, SHANGHAI (CN)

(21) Appl. No.: 17/737,197

(22) Filed: May 5, 2022

(30) Foreign Application Priority Data
May 28, 2021 (CN) 202110594734.7

(51) Int. Cl. G16H 50/80

(52) U.S. Cl. CPC G16H 50/80

(57) ABSTRACT

Proposed are concepts for generating recommendations of disease-related resources in different geographical regions. In particular, it is proposed to leverage information about the popularity (e.g. frequency, rate or regularity of use) of keywords associated with diseases across different geographic regions. Using such information, a relevancy value (i.e. geography weighting score score) for a disease-related resource with respect to a specific geographic region may be calculated. These relevance values may then be used to generate recommendations for the disease-related resources for the specific geographic region.

### CROSS-REFERENCE TO PRIOR APPLICATIONS

This application claims the benefit of Chinese Patent Application No. 202110594734.7, filed May 28, 2021 which is hereby incorporated by reference herein.

### FIELD OF THE INVENTION

The invention relates to the medical domain and more particularly to recommendation of disease-related resources to users.

### BACKGROUND OF THE INVENTION

In the medical domain, there is a general desire to provide disease-related resources and/or content to users according to their needs or desires. That is, personalized recommendation of disease-related resources is generally desirable.

Existing personalized recommendation approaches include collaborative filtering or content filtering, or a combination of both. However, the results of such approaches are typically not timely and/or not effective. This can result in reduced levels of user engagement with educational content and/or inappropriate allocation of medical resources.

Accordingly, there remains a need for improved personalized recommendation of disease-related resources.

### SUMMARY OF THE INVENTION

The invention is defined by the claims.

In accordance with one aspect, there is provided a method for recommendation of disease-related resources in different geographical regions. The method comprises: identifying, from disease-related resource documents, a plurality of keywords associated with a plurality of diseases respectively; determining trend indexes for the plurality of diseases in a geographic region based on popularity of the identified keywords in the geographic region, wherein the trend index for a disease is indicative of relevancy of the disease in the geographic region; calculating a geography weighting score for disease-related resources in the geographic region based on the determined trend indexes; and generating a recommendation of disease-related resources based on the calculated geography weighting score.

By way of example, proposed concepts may leverage information about trends in diseases across different geographic regions. That is, embodiments propose to use a spatial feature, geographic region, based on a realisation that disease trends in different areas may vary (e.g. a northern region and the southern region may have very different flu trends in the same season). In this way, embodiments may facilitate the recommendation of disease-related resources during (or before) a new disease trend develops.

By way of example, proposed embodiments may employ keywords and the (disease) trend indexes in different geographic regions to determine the relevance disease-related resource to each the different geographic regions. The determined relevance of such a resource to a geographic region may then be used to influence recommendation of the resource for that geographic region. Embodiments may thus facilitate medical resource allocation guidance for different geographic regions.

Data mining concepts based on regional disease trends are therefore proposed which may recommend the most relevant disease-related resources (e.g. educational content or medical resources) in each of a plurality of different geographic regions. Improved recommendation of learning materials for healthcare workers may therefore be provided by proposed embodiments. Additionally, or alternatively, embodiments may facilitate improved guidance for the medical resource allocation within different geographic regions.

In some embodiments, the geography weighting score may comprise a vector representing relevancy of disease-related resources to the determined trend indexes in the geographic region. For instance, the weighting score may be a weighted vector that reflects how relevant each disease-related resource is to a recent disease trend.

The disease-related resources may comprise a plurality of educational documents (i.e. educational content) or medical resources at a medical facility. Further, the disease-related resource documents may comprise a description of the educational contents or the medical resources at the medical facility respectively. By way of example, embodiments may employ natural language processing algorithms and/or machine-learning algorithms to identify/extract keywords within/from written descriptions of: (medical-related) educational subject-matter; and/or medical resources.

In some embodiments, determining a trend index for a disease in a geographic region may comprise analysing statistical data about the provision of one or more of the identified keywords to a search engine. Internet-based search engines may therefore be leveraged to facilitate statistical analysis for identifying the popularity (i.e. relevance) of keywords (and thus disease trends).

Generating a recommendation of disease-related resources may be further based on a user interest. In this way, embodiments may take account of user preferences or choices.

In some embodiments, calculating a geography weighting score for disease-related resources in the geographic region may comprise: identifying which of the plurality of diseases each of the disease-related resources relate to; and for each of the disease-related resources, calculating a geography weighting score based on the determined trend index for each of diseases the disease-related resource is identified as relating to.

For example, calculating a geography weighting score for disease-related resources in the geographic region may comprise: representing the disease-related resources and diseases each of the disease-related resources relate to as a first matrix; representing the plurality of diseases and the determined trend indexes for the plurality of diseases as a second matrix; and performing a matrix multiplication with the first matrix and the second matrix to obtain a vector representing the geography weighting score for each of the disease-related resources. That is, embodiments may perform matrix multiplication to obtain a weighted score vector indicating how relevant each disease-related resources is to a disease trend for a geographic region.

According to another aspect of the invention, there is provided a computer program product comprising computer program code means which, when executed on a computing device having a processing system, cause the processing system to perform all of the steps of the method according to a proposed embodiment. Thus, according to examples in accordance with an aspect of the invention, there is provided a computer program comprising code means for implementing the method according to a proposed embodiment when said program is run on a processing system.

According to another aspect of the invention, there is provided a system for recommending disease-related resources in different geographical regions. The system comprises: a keywords extractor configured to identify, from disease-related resource documents, a plurality of keywords associated with a plurality of diseases respectively; a trend index generator configured to determine trend indexes for the plurality of diseases in a geographic region based on popularity of the identified keywords in the geographic region, wherein the trend index for a disease is indicative of relevancy of the disease in the geographic region; a calculation unit configured to calculate a geography weighting score for disease-related resources in the geographic region based on the determined trend indexes; and a recommendation unit configured to generate a recommendation of disease-related resources based on the calculated geography weighting score.

Proposed embodiments may provision a geographical-dependent resource recommendation system. Also, embodiments may provide for personalized (i.e. user-specific) disease-related resource recommendations. Embodiments may therefore be of particular use/benefit in e-learning platforms and/or hospital resource allocation systems.

Still further advantages of the present invention will be appreciated to those of ordinary skill in the art upon reading and understand the following detailed description.

These and other aspects of the invention will be apparent from and elucidated with reference to the embodiments described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 is a simplified flow diagram of a method for recommendation of disease-related resources in different geographical regions according to an embodiment

FIG. 2 is a is a simplified block diagram of a system according to an embodiment

FIG. 3 is a graph depicting an exemplary variation of trend index values output from the trend index generator of FIG. 2 over a time period.

FIG. 4A illustrates an exemplary application of the system of FIG. 2.

FIG. 4B illustrates another exemplary application of the system of FIG. 2.

FIG. 5 is simplified block diagram of a computer within which one or more parts of an embodiment may be employed.

### DETAILED DESCRIPTION OF THE EMBODIMENTS

The invention will be described with reference to the Figures.

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the Figures to indicate the same or similar parts.

The invention provides concepts for generating recommendations of disease-related resources in different geographical regions. In particular, it is proposed to leverage information about the popularity (e.g. frequency, rate or regularity of use) of keywords associated with diseases across different geographic regions. Using such information, a relevancy value (i.e. geography weighting score score) for a disease-related resource with respect to a specific geographic region may be calculated. These relevance values may then be used to generate recommendations for the disease-related resources for the specific geographic region.

Proposed embodiments may therefore be employed to provide geographic-dependent trend-aware recommendations that help to improve allocation of disease-related resources across different geographic regions.

Proposed embodiments may thus, for example, facilitate generation of personalized/customized recommendations disease-related resources that are tailored/adapted to specific geographic regions (e.g. towns, cities, postal codes, states, counties, countries, continents, etc.). Information about disease trends in different regions may therefore be used to provide refined disease-related resource recommendations.

According to a concept of the invention, there is proposed a method and system for recommendation of disease-related resources in different geographical regions. Keywords associated with diseases respectively are extracted from disease-related resource documents. Trend indexes for the diseases in a geographic region are then determined based on popularity of the identified keywords in the geographic region. Geography weighting score for disease-related resources in the geographic region are then calculated based on the determined trend indexes. Using the calculated geography weighting score, a recommendation of disease-related resources can be generated.

Illustrative embodiments may, for example, be employed in relation to healthcare-related digital services, include medical training/educational platform and/or medical resource allocation systems.

Implementations in accordance with the present disclosure relate to various systems, adaptions, and/or methods for recommending disease-related resources in different geographical regions. According to proposed concepts, a number of possible solutions may be implemented separately or jointly. That is, although these possible solutions may be described below separately, two or more of these possible solutions may be implemented in one combination or another.

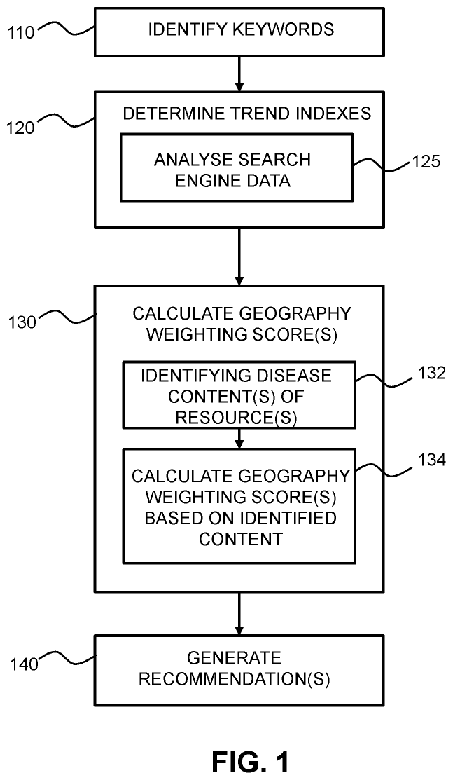

By way of example, a first simplified embodiment will now be described with reference to FIG. 1.

FIG. 1 is a simplified flow diagram of a method for recommendation of disease-related resources in different geographical regions according to an embodiment. Here, the disease-related resources comprise a plurality of educational documents E.g. documents comprising medical-related content) or descriptions of medical resources at a one or more medical facilities.

The method begins with the step 110 of identifying, from disease-related resource documents, a plurality of keywords associated with a plurality of diseases respectively. By way of example, step 110 of identifying keywords may employ one or more of a plurality of known Natural Language Processing (NLP) algorithms. Step 110 of identifying keywords may alternatively (or additionally) comprise providing the disease-related resource documents to a machine-learning algorithm (e.g. neural network) to obtain one or more keywords as prediction results from the machine-learning algorithm. In this way, keywords may be identified/extracted from written descriptions of (medical-related) subject-matter and/or medical resources.

Next, in step 120, trend indexes for the plurality of diseases in a geographic region are determined based on popularity of the identified keywords in the geographic region. A trend index for a disease comprises a value indicative of a relevancy (e.g. popularity, prevalence or commonness) of the disease in the geographic region. In the example of FIG. 1, determining a trend index for a disease in a geographic region comprises the step 125 of analysing statistical data about the provision of one or more of the identified keywords to a search engine. Internet-based search engines are thus leveraged to facilitate statistical analysis for identifying the relevance of the keywords.

The trend indexes determined in step 120 are then used in step 130 to calculate a geography weighting score for disease-related resources in the geographic region. Here, a geography weighting score comprises a vector representing relevancy of disease-related resources to the determined trend indexes in the geographic region.

More specifically, in the example embodiment of FIG. 1, the step 130 of calculating a geography weighting score for disease-related resources in the geographic region comprises two sub-steps, 132 and 134. Step 132 comprises identifying which of the plurality of diseases each of the disease-related resources relate to (e.g. using a NLP algorithm). Step 134 comprises, for each of the disease-related resources, calculating a geography weighting score based on the determined trend index for each of diseases the disease-related resource is identified as relating to. More specifically, in this example embodiment, calculating a geography weighting score for disease-related resources in the geographic region comprises: representing the disease-related resources and diseases each of the disease-related resources relate to as a first matrix; representing the plurality of diseases and the determined trend indexes for the plurality of diseases as a second matrix; and performing a matrix multiplication with the first matrix and the second matrix. In this way, the result of the matrix multiplication provides a vector representing the geography weighting score for each of the disease-related resources.

Finally, in step 140, a recommendation of disease-related resources is generated based on the calculated geography weighting score.

It is to be understood that the method described above with reference to FIG. 1 is not restricted to the specific ordering of the step described/illustrated.

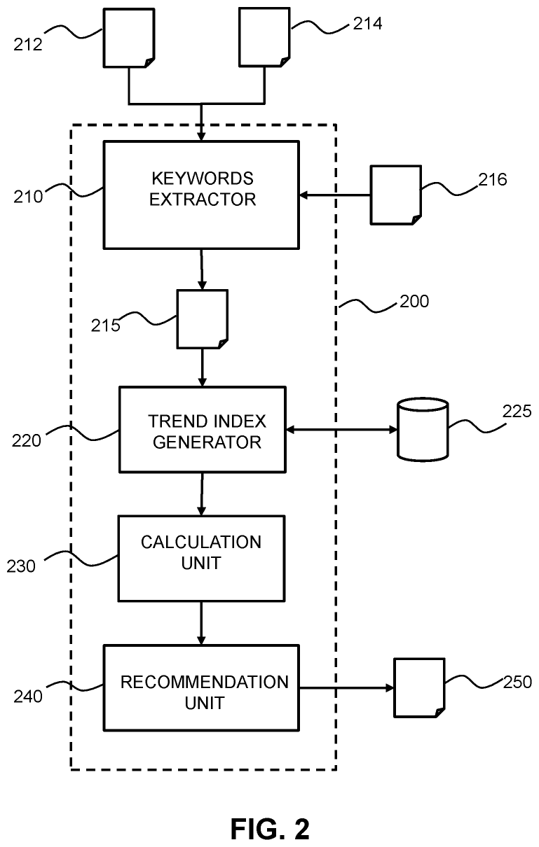

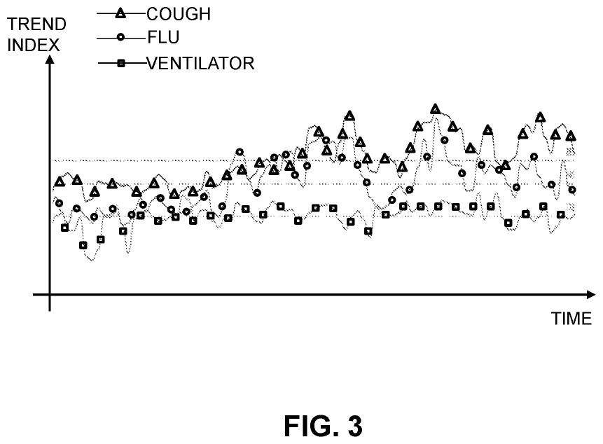

By way of further example and explanation, an exemplary embodiment of a system for recommending disease-related resources in different geographical regions will now be described with reference to FIGS. 2 and 3.

FIG. 2 is a simplified block diagram of a system 200 according to an embodiment. The system 200 may be used for creating packaged learning materials or hospital resource allocation guidance by using components such as a keywords extractor 210 and a trend index generator 220.

The keywords extractor 210 is configured to identify, from disease-related resource documents, a plurality of keywords associated with a plurality of diseases respectively. Specifically, in the example embodiment of FIG. 2, the disease-related resources documents comprise contents/learning materials 212 from an e-learning platform or medical resource information 214 about the hospitals (e.g. departments, size, and city). These documents 212 or 214 are provided as inputs to the keywords extractor 210. Also provided as an input to the keywords extractor 210 is a list of diseases 216 identifying a plurality of diseases of interest.

The keywords extractor 210 can be realized by a known NLP algorithm (e.g. TF-IDF, Co-occurrence, and deep learning approaches) combined with expert (i.e. supervisor) experience.

The keywords extractor 210 generates and outputs a list of keywords 215 related to the diseases of interest, which may, for example, comprise ‘cough’, ‘flu’, ‘fever’, ‘COPD’, ‘NIV’, etc.

The list of keywords 215 is provided to the trend index generator 220 of the system 200. The trend index generator 220 is configured to determine trend indexes for the plurality of diseases in a geographic region based on popularity of the identified keywords in the geographic region. In particular, the trend index for a disease is indicative of relevancy of the disease in the geographic region. Determined trend indexes can therefore summarize the popularity of the keywords/diseases in different regions.

The trend index generator 220 may be implemented by leveraging data from a database 225 or search engine. For instance, the trend index generator 220 may employ a data analysis unit which undertakes statistical analysis about the provision of one or more of the identified keywords to a database or search engine in order to determine the popularity of keywords.

FIG. 3 depicts an example of the outputs from the trend index generator with respect to time. Specifically, FIG. 3 is a graph depicting an exemplary variation of trend index values output from the trend index generator 220 over a time period. It depicts the varying popularity of three keywords: ‘cough’ (the lined having triangle markers); ‘flu’ (the line having circle markers); and ‘ventilator’ (the lien having square markers) over a time period of three months in a city (e.g. Shanghai). The dashed lines represent the average popularity of each keyword, respectively.

The exemplary trend index data of FIG. 3 indicates that the ventilator has stable popularity in this period in the city. From this is may be inferred that no change in strategy related to this topic is required for the city. For the flu index, there is a steady increase in the trend index (i.e. popularity) for the first two months of the three-month period, and then a decline in the final month of the three-month period (ending below its average value). Based on this is may be inferred that resources related to the flu may be slightly reduced. The cough trend index has a similar trend with the flu trend index but shows a slight increase at the end of the three month period (with the endpoint being above its average value). From this, it may be inferred that the related departments may need more resources. In addition to this, for e-learning platforms, it may be valuable for to recommend more flu-related courses/materials for the healthcare workers (e.g. to be prepared for the latest flu trend).

Referring back to FIG. 2, the determined trend indexes are provided from the trend index generator 220 to the calculation unit 230 of the system 200. The calculation unit calculates a geography weighting score for disease-related resources in the geographic region based on the determined trend indexes. For example, the calculation unit 230 in this embodiment is configured to calculate a vector representing relevancy of disease-related resources to the determined trend indexes in the geographic region.

The calculated geography weighting scores are provided to a recommendation unit 240 which is configured to generate a recommendation (e.g. list) 250 of disease-related resources based on the geography weighting scores Such recommendations may, for example, be generated based on inferences such as those detailed above with reference to the exemplary data of FIG. 3. In doing so, a user input representing user interest may also be accounted for.

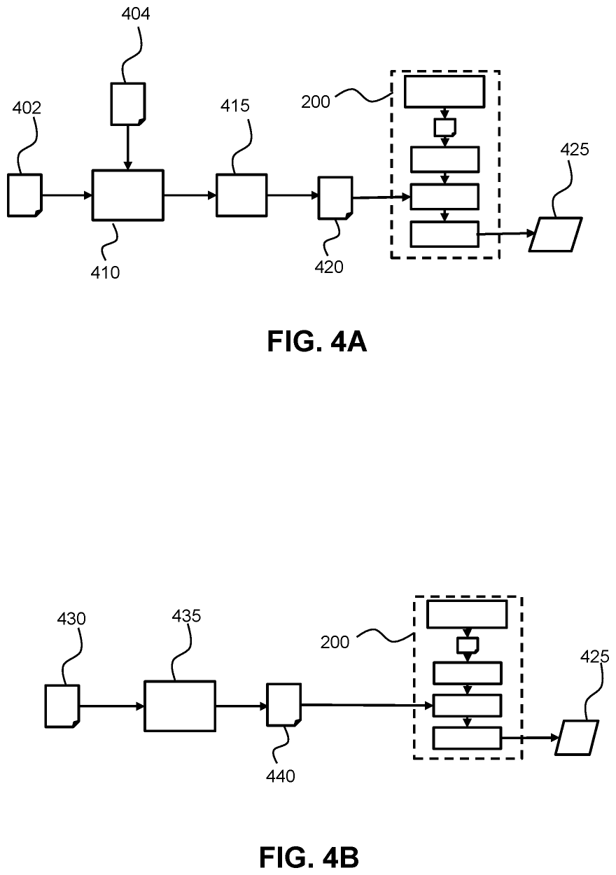

Referring now to FIGS. 4A and 4B, exemplary applications/uses of the system 200 of FIG. 2 are illustrated.

In the first exemplary application (depicted in FIG. 4A), user information 402 and available courses 404 are provided to an embedding module 410. The embedding module embeds the user information 402 and available courses 404 to create a model 415 for recommending learning courses for a user. This model 415 may employ a recurrent neural network for generating a prediction 420 of the most popular learning courses (e.g. an initial recommendation list). The prediction 420 is then provided to the system 200 to combine with the obtained disease trend indexes. In doing so, a weighted vector 425 may be constructed which represents how relevant a course is according to recent disease trends.

For example, a matrix representation of the disease-related resources (e.g. Learning Courses) and diseases each of the disease-related resources relate may provided as a first matrix as follows:

| | Flu | Cough | Ventilator |
|---|---|---|---|
| Course A | 1 | 1 | 0 |
| Course B | 0 | 0 | 1 |
| Course C | 1 | 0 | 1 |
| Course D | 0 | 1 | 0 |
| Course E | 1 | 1 | 1 |

The above first matrix indicates a one-hot encoding map to represent whether a course is related to each keyword—if yes it is 1, else it is 0. Alternatively, the matrix can be achieved by a machine-learning algorithm, such as an NLP-algorithm can determine which topics are related to the content and even a relevance score can be predicted rather than the 0 and 1 here.

Also, an exemplary matrix representation of the plurality of diseases and the determined trend indexes for the plurality of diseases may be provided as a second (score) matrix as follows:

| | Popularity Score |
|---|---|
| Flu | 0.4 |
| Cough | 0.38 |
| Ventilator | 0.22 |

The above second matrix represents the normalized popularity score for each keyword.

By performing a matrix multiplication with the first matrix and second matrix, the following weighted score vector is obtained:

| | Weighted Score |
|---|---|
| Course A | 0.78 |
| Course B | 0.22 |
| Course C | 0.62 |
| Course D | 0.38 |
| Course E | 1 |

The weighted score vector above thus indicates how relevant a course in relation to the disease trends. The weighted score vector can therefore be used to modify the prediction 420 (e.g. re-rank the initial recommendation list) so as to provide recommendations based on the disease trends. In this way, the generated recommendations) may not only account for user interest(s) but also account for potential knowledge needs (in view of disease trends).

In the second exemplary application (depicted in FIG. 4B), hospital resources 430 may be provided to a conventional resource allocation system 435 to provide an initial recommendation 440. The initial recommendation 440 is then provided to the system 200 for calculation based on the obtained disease trend indexes. Similarly to the example of FIG. 4A, a weighted vector 425 may be constructed. For instance, the first matrix above may be replaced by the relevance between the different departments and different keywords (wherein one-hot encoding can be replaced with a machine-learning algorithm to have continuous values). Thus, a weighted score vector 425 can be generated which indicate which department of a hospital may need more resources according to disease trends.

Accordingly, it will be understood that the proposed concept(s) may facilitate geographical-dependent resource recommendation. Embodiments may thus provide for personalized (i.e. user-specific) disease-related resource recommendations, wherein the recommendations take account of one or more disease trends in a geographical region.

Proposed methods and system (including those of FIGS. 2 to 4) may be implemented in hardware or software, or a mixture of both (for example, as firmware running on a hardware device). To the extent that an embodiment is implemented partly or wholly in software, the functional steps illustrated in the process flowcharts may be performed by suitably programmed physical computing devices, such as one or more central processing units (CPUs) or graphics processing units (GPUs). Each process—and its individual component steps as illustrated in the flowcharts—may be performed by the same or different computing devices.

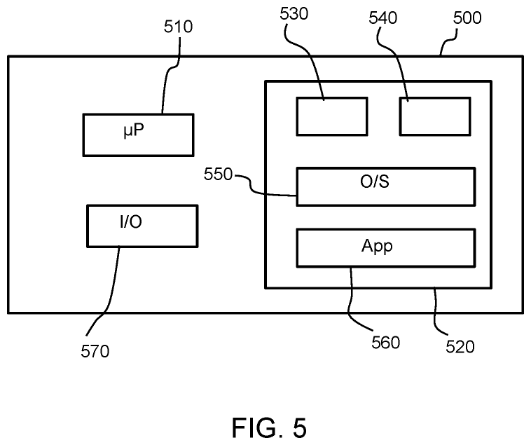

FIG. 5 illustrates an example of a computer 500 within which one or more parts of an embodiment may be employed. Various operations discussed above may utilize the capabilities of the computer 500. For example, one or more parts of a system for for recommending disease-related resources in different geographical regions may be incorporated in any element, module, application, and/or component discussed herein. In this regard, it is to be understood that system functional blocks can run on a single computer or may be distributed over several computers and locations (e.g. connected via internet).

The computer 500 includes, but is not limited to, PCs, workstations, laptops, PDAs, palm devices, servers, storages, and the like. Generally, in terms of hardware architecture, the computer 500 may include one or more processors 510, memory 520, and one or more I/O devices 570 that are communicatively coupled via a local interface (not shown). The local interface can be, for example but not limited to, one or more buses or other wired or wireless connections, as is known in the art. The local interface may have additional elements, such as controllers, buffers (caches), drivers, repeaters, and receivers, to enable communications. Further, the local interface may include address, control, and/or data connections to enable appropriate communications among the aforementioned components.

The processor 510 is a hardware device for executing software that can be stored in the memory 520. The processor 510 can be virtually any custom made or commercially available processor, a central processing unit (CPU), a digital signal processor (DSP), or an auxiliary processor among several processors associated with the computer 500, and the processor 510 may be a semiconductor based microprocessor (in the form of a microchip) or a microprocessor.

The memory 520 can include any one or combination of volatile memory elements (e.g., random access memory (RAM), such as dynamic random access memory (DRAM), static random access memory (SRAM), etc.) and non-volatile memory elements (e.g., ROM, erasable programmable read only memory (EPROM), electronically erasable programmable read only memory (EEPROM), programmable read only memory (PROM), tape, compact disc read only memory (CD-ROM), disk, diskette, cartridge, cassette or the like, etc.). Moreover, the memory 820 may incorporate electronic, magnetic, optical, and/or other types of storage media. Note that the memory 520 can have a distributed architecture, where various components are situated remote from one another, but can be accessed by the processor 510.

The software in the memory 520 may include one or more separate programs, each of which comprises an ordered listing of executable instructions for implementing logical functions. The software in the memory 520 includes a suitable operating system (O/S) 550, compiler 540, source code 530, and one or more applications 560 in accordance with exemplary embodiments. As illustrated, the application 560 comprises numerous functional components for implementing the features and operations of the exemplary embodiments. The application 560 of the computer 500 may represent various applications, computational units, logic, functional units, processes, operations, virtual entities, and/or modules in accordance with exemplary embodiments, but the application 560 is not meant to be a limitation.

The operating system 550 controls the execution of other computer programs, and provides scheduling, input-output control, file and data management, memory management, and communication control and related services. It is contemplated by the inventors that the application 560 for implementing exemplary embodiments may be applicable on all commercially available operating systems.

Application 560 may be a source program, executable program (object code), script, or any other entity comprising a set of instructions to be performed. When a source program, then the program is usually translated via a compiler (such as the compiler 540), assembler, interpreter, or the like, which may or may not be included within the memory 520, so as to operate properly in connection with the O/S 550. Furthermore, the application 560 can be written as an object oriented programming language, which has classes of data and methods, or a procedure programming language, which has routines, subroutines, and/or functions, for example but not limited to, C, C++, C#, Pascal, BASIC, API calls, HTML, XHTML, XML, ASP scripts, JavaScript, FORTRAN, COBOL, Perl, Java, ADA, .NET, and the like.

The I/O devices 570 may include input devices such as, for example but not limited to, a mouse, keyboard, scanner, microphone, camera, etc. Furthermore, the I/O devices 570 may also include output devices, for example but not limited to a printer, display, etc. Finally, the I/O devices 570 may further include devices that communicate both inputs and outputs, for instance but not limited to, a NIC or modulator/demodulator (for accessing remote devices, other files, devices, systems, or a network), a radio frequency (RF) or other transceiver, a telephonic interface, a bridge, a router, etc. The I/O devices 570 also include components for communicating over various networks, such as the Internet or intranet.

If the computer 500 is a PC, workstation, intelligent device or the like, the software in the memory 520 may further include a basic input output system (BIOS) (omitted for simplicity). The BIOS is a set of essential software routines that initialize and test hardware at startup, start the O/S 550, and support the transfer of data among the hardware devices. The BIOS is stored in some type of read-only-memory, such as ROM, PROM, EPROM, EEPROM or the like, so that the BIOS can be executed when the computer 500 is activated.

When the computer 500 is in operation, the processor 510 is configured to execute software stored within the memory 520, to communicate data to and from the memory 520, and to generally control operations of the computer 500 pursuant to the software. The application 560 and the O/S 550 are read, in whole or in part, by the processor 510, perhaps buffered within the processor 510, and then executed.

When the application 560 is implemented in software it should be noted that the application 560 can be stored on virtually any computer readable medium for use by or in connection with any computer related system or method. In the context of this document, a computer readable medium may be an electronic, magnetic, optical, or other physical device or means that can contain or store a computer program for use by or in connection with a computer related system or method.

The application 560 can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable medium” can be any means that can store, communicate, propagate, or transport the program for use by or in connection with the instruction execution system, apparatus, or device. The computer readable medium can be, for example but not limited to, an electronic, magnetic, optical, electromagnetic, infrared, or semiconductor system, apparatus, device, or propagation medium.

The present invention may be a system, a method, and/or a computer program product. The computer program product may include a computer readable storage medium (or media) having computer readable program instructions thereon for causing a processor to carry out aspects of the present invention.

According to embodiments, a computer-readable storage medium stores a computer program comprising computer program code configured to cause one or more physical computing devices to carry out a method as described above when the program is run on the one or more physical computing devices.

Storage media may include volatile and non-volatile computer memory such as RAM, PROM, EPROM, and EEPROM. Various storage media may be fixed within a computing device or may be transportable, such that the one or more programs stored thereon can be loaded into a processor.

All of part of a schematic model according to an embodiment may be stored on a storage medium. Data according to an embodiment may be stored on the same storage medium or a different storage medium. The schematic model and/or data may be transmitted as a signal modulated onto an electromagnetic carrier wave. The signal may be defined according to a standard for digital communications. The carrier wave may be an optical carrier, a radio-frequency wave, a millimeter wave, or a near field communications wave. It may be wired or wireless.

To the extent that an embodiment is implemented partly or wholly in hardware, the blocks shown in the diagrams of FIGS. 2, 4 and 5 may be separate physical components, or logical subdivisions of single physical components, or may be all implemented in an integrated manner in one physical component. The functions of one block shown in the drawings may be divided between multiple components in an implementation, or the functions of multiple blocks shown in the drawings may be combined in single components in an implementation. Hardware components suitable for use in embodiments of the present invention include, but are not limited to, conventional microprocessors, application specific integrated circuits (ASICs), and field-programmable gate arrays (FPGAs). One or more blocks may be implemented as a combination of dedicated hardware to perform some functions and one or more programmed microprocessors and associated circuitry to perform other functions.

As used herein, a memory includes one or more of a non-transient computer readable medium; a magnetic disk or other magnetic storage medium; an optical disk or other optical storage medium; a random access memory (RAM), read-only memory (ROM), or other electronic memory device or chip or set of operatively interconnected chips; an Internet/Intranet server from which the stored instructions may be retrieved via the Internet/Intranet or a local area network; or so forth. Further, as used herein, a processor includes one or more of a microprocessor, a microcontroller, a graphic processing unit (GPU), an application-specific integrated circuit (ASIC), a field-programmable gate array (FPGA), and the like; a user input device includes one or more of a mouse, a keyboard, a touch screen display, one or more buttons, one or more switches, one or more toggles, and the like; a display device includes one or more of a liquid crystal display (LCD), an light emitting diode (LED) display, a plasma display, a projection display, a touch screen display, and the like; and databases include one or more memories.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

It will also be understood that the disclosed methods may be computer-implemented methods. As such, there is also proposed a concept of a computer program comprising code means for implementing any described method when said program is run on a processing system.

The skilled person would be readily capable of developing a processor for carrying out any herein described method. Thus, each step of a flow chart may represent a different action performed by a processor, and may be performed by a respective module of the processing processor.

A proposed system may make use of a processor to perform data processing. The processor can be implemented in numerous ways, with software and/or hardware, to perform the various functions required. The processor typically employs one or more microprocessors that may be programmed using software (e.g. microcode) to perform the required functions. The processor may be implemented as a combination of dedicated hardware to perform some functions and one or more programmed microprocessors and associated circuitry to perform other functions.

Examples of circuitry that may be employed in various embodiments of the present disclosure include, but are not limited to, conventional microprocessors, application specific integrated circuits (ASICs), and field-programmable gate arrays (FPGAs).

In various implementations, the processor may be associated with one or more storage media such as volatile and non-volatile computer memory such as RAM, PROM, EPROM, and EEPROM. The storage media may be encoded with one or more programs that, when executed on one or more processors and/or controllers, perform the required functions. Various storage media may be fixed within a processor or controller or may be transportable, such that the one or more programs stored thereon can be loaded into a processor.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. If the term “adapted to” is used in the claims or description, it is noted that the term “adapted to” is intended to be equivalent to the term “configured to”. Any reference signs in the claims should not be construed as limiting the scope.

1. A method for recommendation of disease-related resources in different geographical regions, the method comprising:
identifying, from disease-related resource documents, a plurality of keywords associated with a plurality of diseases respectively;
determining trend indexes for the plurality of diseases in a geographic region based on popularity of the identified keywords in the geographic region, wherein the trend index for a disease is indicative of relevancy of the disease in the geographic region;
calculating a geography weighting score for disease-related resources in the geographic region based on the determined trend indexes; and
generating a recommendation of disease-related resources based on the calculated geography weighting score.

2. The method of claim 1, wherein the geography weighting score comprises a vector representing relevancy of disease-related resources to the determined trend indexes in the geographic region.

3. The method of claim 1, wherein the disease-related resources comprises a plurality of educational contents or medical resources at a medical facility, and wherein the disease-related resource documents comprise a description of the educational contents or the medical resources at the medical facility respectively.

4. The method of claim 1, wherein determining a trend index for a disease in a geographic region comprises:
analysing statistical data about the provision of one or more of the identified keywords to a search engine.

5. The method of claim 1, wherein generating a recommendation of disease-related resources is further based on a user interest.

6. The method of claim 1, wherein calculating a geography weighting score for disease-related resources in the geographic region comprises:
identifying which of the plurality of diseases each of the disease-related resources relate to; and
for each of the disease-related resources, calculating a geography weighting score based on the determined trend index for each of diseases the disease-related resource is identified as relating to.

7. The method of claim 6, wherein calculating a geography weighting score for disease-related resources in the geographic region comprises:
representing the disease-related resources and diseases each of the disease-related resources relate to as a first matrix;
representing the plurality of diseases and the determined trend indexes for the plurality of diseases as a second matrix; and
performing a matrix multiplication with the first matrix and the second matrix to obtain a vector representing the geography weighting score for each of the disease-related resources.

8. A computer program product comprising computer program code means which, when executed on a computing device having a processing system, cause the processing system to perform all of the steps of the method according to claim 1.

9. A system for recommending disease-related resources in different geographical regions, the system comprising:
a keywords extractor configured to identify, from disease-related resource documents, a plurality of keywords associated with a plurality of diseases respectively;
a trend index generator configured to determine trend indexes for the plurality of diseases in a geographic region based on popularity of the identified keywords in the geographic region, wherein the trend index for a disease is indicative of relevancy of the disease in the geographic region;
a calculation unit configured to calculate a geography weighting score for disease-related resources in the geographic region based on the determined trend indexes; and
a recommendation unit configured to generate a recommendation of disease-related resources based on the calculated geography weighting score.

10. The system of claim 9, wherein the calculation unit is configured to calculate a vector representing relevancy of disease-related resources to the determined trend indexes in the geographic region.

11. The system of claim 9, wherein the disease-related resources comprises a plurality of educational contents or medical resources at a medical facility, and wherein the disease-related resource documents comprise a description of the educational contents or the medical resources at the medical facility respectively.

12. The system of claim 9, wherein trend index generator comprises:
a data analysis unit configured to analyse statistical data about the provision of one or more of the identified keywords to a search engine.

13. The system of claim 9, wherein the recommendation unit is configured to generate a recommendation of disease-related resources further based on a user interest.

14. The system of claim 9, wherein the calculation unit is configured to:
identify which of the plurality of diseases each of the disease-related resources relate to; and
for each of the disease-related resources, calculate a geography weighting score based on the determined trend index for each of diseases the disease-related resource is identified as relating to.

15. The system of claim 14, wherein the calculation unit comprises:
a matrix generator configured to represent the disease-related resources and diseases each of the disease-related resources relate to as a first matrix;
a vector generator configured to represent the plurality of diseases and the determined trend indexes for the plurality of diseases as a second matrix; and
a multiplication unit configured to perform a matrix multiplication with the first matrix and the second matrix to obtain a vector representing the geography weighting score for each of the disease-related resources.
